(19) United States
(12) Patent Application Publication
(10) Pub. No.: US 2014/0323879 A1
(43) Pub. Date: Oct. 30, 2014

(54) SYSTEMS, DEVICES AND METHODS FOR MONITORING HEMODYNAMICS

(71) Applicant: Radiation Monitoring Devices, Inc., Watertown, MA (US)

(72) Inventors: Madhavi Seetamraju, Lexington, MA (US); Rajan S. Gurjar, Arlington, MA (US); David E. Wolf, Sudbury, MA (US)

(73) Assignee: Radiation Monitoring Devices, Inc., Watertown, MA (US)

(21) Appl. No.: 14/222,725

(22) Filed: Mar. 24, 2014

Related U.S. Application Data
(62) Division of application No. 13/092,539, filed on Apr. 22, 2011.
(60) Provisional application No. 61/433,915, filed on Jan. 18, 2011.

(51) Int. Cl. A61B 5/026

(52) U.S. Cl. CPC A61B 5/0261
USPC 600/479

(57) ABSTRACT

Systems, devices and methods for monitoring hemodynamics are described. The systems and methods generally involve directing light toward an area of the body and detecting the resulting scattered light. The scattered light is detected and an electrical signal representative of the scattered light intensity is generated from the detected light. The electrical signal is analyzed by measuring temporal fluctuations of such signals to monitor pathological states over time including hemorrhagic shock, hypoxia, and tissue graft vascularization. Such monitoring can have significant benefits to patients.

### RELATED APPLICATIONS

This application is a divisional of U.S. patent application Ser. No. 13/092,539, filed Apr. 22, 2011, which claims priority to U.S. Provisional Patent Application Ser. No. 61/433,915, filed Jan. 18, 2011, which are incorporated herein by reference in their entirety.

### FIELD OF INVENTION

The invention relates generally to the field of monitoring hemodynamics as a means of monitoring the onset, progression, or regression of physiological or pathological conditions.

### BACKGROUND OF INVENTION

In general, monitoring the onset, progression or regression of certain physiological or pathological conditions is important in the treatment of patients. These conditions include hemorrhagic shock, tissue graft vascularization and hypoxia.

Hemorrhagic shock results from decreased cardiac output and the resultant drop in intravascular volume (hypovolemia). However, in emergency departments “shock is typically recognized by non-specific signs and subjective symptoms such as: cold clammy skin, pallor, weak thready pulse, unstable vital signs, and diminished mentation. Unfortunately, these signs are imprecise, subjective, and inconsistent. Consequently, there has been considerable effort to develop noninvasive shock monitors based on, for instance, gastric or sublingual pH measurement, near-infrared reflectance oximetry, beat-to beat heart rate variability, and acoustic arterial flow analysis. However, even these physiological parameters occur too late in the sequence of physiological responses to shock to be used as early indicators of the onset of life threatening hemorrhagic shock. Very similar delays are seen with experimental technologies such as NIR measurement of tissue pO2 and a reliable early predicting system has yet to be developed. A system that could reliably indicate the onset of hemorrhagic shock would save thousands of lives.

Modern tissue grafting techniques often involve a four step process: construction of a suitable scaffold for tissue growth, seeding and growth of cells into the scaffold in tissue culture, implantation of the graft into a buried flap for instance in the arm or back of the patient to enable vascularization of the tissue, and transplantation of the graft to its final site. This process enables recreation through tissue engineering of complex multilaminar tissues by tissue engineering. Both the processes of buried flap vascularization and final grafting are dependent upon proper capillary blood perfusion and monitoring such conditions can be important to patient treatment.

Pressure ulcers, represent a significant problem in nursing homes and hospitals. It is estimated that 2.5 million pressure ulcerations are treated each year with a cost to the healthcare system of $11 billion. Treatment of a pressure ulcer ranges from $500 to $40,000 depending upon severity. Pressure ulcerations result from a variety of conditions including: unconsciousness, quadriplegia, long-term confinement to beds or wheelchairs, and prolonged surgery. Approximately 2% of patients hospitalized for other conditions develop pressure ulcer and 11.6% of these people die, which is 4.5 fold greater mortality rate than for patients who do not develop pressure ulcers. Pressure ulcerations cause ˜60,000 horribly painful deaths per year in the United States. At the same time, the vast majority of pressure ulcers are preventable if detected before damage occurs.

Deep tissue injury results in severe deformation causing tissue damage or pressure-induced hypoxia leading to ischemia. If deformations are severe and exceed a threshold value, rapid tissue damage, such as cellular or blood vessel collapse, can occur. Often this results from frictional shear at the soft tissue bone interface, where there are force components both normal and parallel to the bone. At lower deformation levels a more gradual ischemic process can occur as a result of hypoxia, glucose depletion, and tissue acidification. Hypoxia is the loss of oxygen to the tissue as a result of loss of tissue blood perfusion.

### SUMMARY OF INVENTION

Systems, devices and methods for monitoring hemodynamics are described.

In one aspect, a method for monitoring hemorrhagic shock of a patient is provided. The method comprises directing light toward a region of a patient including tissue in which blood flows and detecting light scattered by the tissue and the blood. The method further comprises generating a signal representative of the scattered light intensity and analyzing temporal fluctuations in the signal to monitor for hemorrhagic shock in the patient.

In another aspect, a method for monitoring tissue graft vascularization is provided. The method comprises directing light toward a tissue graft and detecting light scattered by the tissue graft. The method further comprises generating a signal representative of the scattered light intensity and analyzing temporal fluctuations in the signal to monitor tissue graft vascularization. In some embodiments, the tissue graft is implanted in a buried flap of a patient; and, in other embodiments, the tissue graft is grafted to a patient.

In another aspect, a method for measuring hypoxia at an interface between soft tissue and bone of a patient is provided. The method comprises directing light toward an interface between the soft tissue the bone of the patient and detecting light scattered by the soft tissue. The method further comprises generating a signal representative of the scattered light intensity and analyzing temporal fluctuations in the signal to measure hypoxia at an interface between soft tissue and bone of the patient.

In some embodiments, the light is directed toward the region of the patient using a fiber optic. In other embodiments, the source of the light is in direct contact with the patient. The source of the light may, for example, be a laser.

In some embodiments, the light is transmitted through the tissue and the blood to produce the scattered light; while, in other embodiments, the light is reflected by the tissue and the blood to produce the scattered light.

In some embodiments, the scattered light is transmitted to a detector using a fiber optic. For example, the scattered light can be transmitted to a detector using a single mode fiber optic. In some embodiments, the detector of the scattered light is in direct contact with the patient.

In some embodiments, the method further comprises wirelessly transferring the signal representative of the scattered light to a processor for analyzing temporal fluctuations in the signal.

The temporal fluctuations in the signal may be representative of changes in blood flow. The method may further comprise analyzing the temporal fluctuations in the signal along with analyzing other physiological data obtained from the patient (e.g., using multiparametric analysis) to monitor for hemorrhagic shock in the patient.

In some embodiments, the method further comprises directing multiple wavelengths of light toward a region of a patient including tissue in which blood flows and analyzing temporal fluctuations in the signal resulting from respective wavelengths to monitor blood and/or tissue oxygen level. For example, the wavelengths of the light source(s) are chosen to further enable determination of the hemoglobin content of the tissue or of the oxygen saturation of the blood in the tissue.

In some embodiments, the temporal fluctuations in the signal are analyzed using an analysis technique selected from the group consisting of: autocorrelation analysis, Fourier analysis, wavelet analysis and pulse height distribution analysis.

In another aspect, an integrated device for assessing blood flow in tissue of a patient is provided. The device is configured to be mounted to the patient. The device comprises a housing and a light source integrated with the housing. The light source is constructed and arranged to direct light toward a region in the patient including tissue in which blood flows. The devices further comprises a single photon counting light detector integrated with the housing. The light detector is constructed and arranged to detect photons of light scattered by the tissue and the blood.

In some embodiments, the housing comprises a polymeric material. The housing, for example, may have a volume of less than 10 cm³. The housing has an outer surface, and the light source and the light detector may be positioned on the outer surface.

The device may further comprise a battery electrically connected to the light source to provide power to the light source.

The light source may be semiconductor-based. For example, the light source may be an LED or laser diode.

The device can further comprise processing electronics integrated with the light detector.

In some embodiments, the light detector is a CMOS-based device. For example, electronic processing circuitry and/or circuitry that controls power (e.g., a battery) and signal transmission can be incorporated into the CMOS-based device.

In some embodiments, the light detector is chosen from the group consisting of: photomultiplier tubes, charge coupled devices, solid state photomultipliers, silicon photodiodes, avalanche photodiodes and Geiger mode avalanche photodiodes.

In some embodiments, the device further comprises an adhesive on a portion of the outer surface of the device.

In some embodiments, the device further comprises a wireless antenna associated with the detector designed to transmit signals representative of the scattered light intensity.

In another aspect, a system for assessing blood flow in tissue of a patient is provided. The system comprises an integrated device for assessing blood flow in tissue of a patient. The device is configured to be mounted to the patient. The device comprises a housing and a light source integrated with the housing. The light source is constructed and arranged to direct light toward a region in the patient including tissue in which blood flows. The device further comprises a single photon counting light detector integrated with the housing. The light detector is constructed and arranged to detect photons of light scattered by the tissue and the blood. The system further comprises a processor configured to analyze temporal fluctuations in the electrical signal to monitor for hemorrhagic shock in the patient.

Other aspects, embodiments and features of the invention will become apparent from the following detailed description of the invention when considered in conjunction with the accompanying drawings. The accompanying figures are schematic and are not intended to be drawn to scale. For purposes of clarity, not every component is labeled in every figure. Nor is every component of each embodiment of the invention shown where illustration is not necessary to allow those of ordinary skill in the art to understand the invention.

### BRIEF DESCRIPTION OF THE DRAWINGS

FIG. 1 is a schematic layout of the components of a system for monitoring patient hemodynamics according to an embodiment.

FIG. 2 is a schematic layout of the components of a system for monitoring patient hemodynamics according to an embodiment.

FIG. 3A is a schematic layout of the components of a system for monitoring patient hemodynamics in an embodiment using a transmission mode.

FIG. 3B is a schematic layout of the components of a system for monitoring patient hemodynamics in an embodiment using a reflectance mode.

FIG. 4A is a sensor patch and associated fiberoptics according to an embodiment.

FIG. 4B is a sensor patch strapped to wrist of patient according to an embodiment.

FIG. 4C shows the scattered light intensity measured in a finger tip with 1 cm fiber separation distance.

FIG. 4D shows the autocorrelation of intensity data from FIG. 4C showing components due to blood flow, heart beat, and respiration.

FIG. 5 is a schematic of self-contained, battery powered, wireless device for monitoring patient hemodynamics according to an embodiment.

FIG. 6 is a plot of the scattered intensity measured as a function of time from blood flowing through a tube and driven by a peristaltic pump.

FIG. 7 shows the autocorrelation function for the data of FIG. 6.

FIG. 8 shows a plot of the inverse fitted time constants τ₁ and τ₂ at 660 nm and 680 nm for blood flowed through a tube at different pump settings.

FIG. 9 shows an example of autocorrelation data from FIG. 8.

FIG. 10A shows a velocity calibration plot obtained from syringe pump driven blood scatter data as a function of flow rate.

FIG. 10B shows the flow rates determined for peristaltic pump driven data from the fitted flow rate and the calibration plot of FIG. 10A.

FIG. 11 shows six plots showing the increasing frequency of the oscillatory/peristaltic component in the correlation plots.

FIG. 12 shows a schematic of the experimental setup for measuring oxygenation in the reflectance mode using light sources at 660 nm and 980 nm.

FIG. 13A shows intensity at 660 nm as a function of pO₂

FIG. 13B shows intensity at 980 nm as a function of pO₂

FIG. 14 shows the ratio of data from FIGS. 13A &B as a function of pO₂

FIG. 15 shows measurements using the device of FIG. 4 on the finger, temple, and over the carotid artery. Left panels show intensity fluctuations with time. Right panels show corresponding correlation functions.

FIG. 16 shows intensity data with device of FIG. 4 from the carotid artery emphasizing long-time respiratory fluctuations under conditions of normal breathing and hyperventilation.

FIG. 17 shows a theoretical calculation of average depth monitored, z, as a function of source-detector separation distances.

FIG. 18 shows a comparison of autocorrelation from a fingertip with separation distance between excitation (multimode) and detection (single mode) fibers of 2 mm (capillary blood flow) and 1 cm (arterial blood flow).

### DETAILED DESCRIPTION

Systems, devices and methods for monitoring hemodynamics are described. The systems and methods generally involve directing light toward an area of the body and detecting the resulting scattered light. The area of the body can include tissue in which blood flows (or should flow under normal physiological conditions) with the incoming light being scattered by the tissue and blood. The scattered light is detected and an electrical signal representative of the scattered light intensity is generated from the detected light. The electrical signal is analyzed, as described further below, by measuring temporal fluctuations of such signals to monitor pathological states over time including hemorrhagic shock, hypoxia, and tissue graft vascularization. Such monitoring can have significant benefits to patients.

The methods can utilize a diffuse correlation spectroscopy (DCS) technique. DCS is a time-domain approach based on correlations in scattered light intensity fluctuations which are related to the dynamics in the probed volume. In the absence of blood flow, the scattering pattern of light (e.g., coherent laser light) reflected off skin will be a constant speckle pattern. In the presence of blood flow, reflections off the moving blood cells contribute to this speckle pattern, resulting in a speckle pattern that fluctuates in time at a frequency characteristic of the movement. Intensity fluctuations are caused not only by net blood flow (μs to ms) but also by to the pulsatile nature of the flow (heart rate in sec), and pulsatile variations due to respiration (10's of sec). DCS involves measuring these fluctuations. Some techniques involve calculating the intensity autocorrelation of the time-series signal. The resulting autocorrelation signal is essentially the average correlation coefficient between the intensity from a speckle at any time and the intensity at some interval in time later.

Autocorrelation analysis is closely related to Fourier analysis. The power spectrum of the signal (as in Doppler measurements) is the Fourier transform of its autocorrelation function. Correlation analysis can have several advantages over Fourier analysis: 1. It is easy to implement in either hardware or software, 2. It can analyze signals over seven to ten orders of magnitude simultaneously, and 3. Because of the way it is calculated, it is essentially an averaging technique despite its high temporal resolution, and therefore improves precision.

In some embodiments, the present invention enables direct measure of blood compensation with time and will significantly improve patient outcome. It may function as a stand-alone indicator of hemorrhagic shock onset or in conjunction with other physiologic monitors to improve the accuracy of smart multiparametric algorithms. In some embodiments, methods of the invention use an optical capillary blood flow measurement. There is widespread agreement on the critical role played by capillary blood flow and resultant tissue perfusion in the etiology of hemorrhagic shock. The body's first response to a hemorrhage is to attempt to form a clot at the site of the bleeding. As hemorrhage continues the body releases catecholamines and antidiuretic hormone in an attempt to maintain blood pressure and tissue oxygenation. Atrial natriuretic receptors increase blood flow resistance by vasoconstriction of the muscle arteries and the arterioles that supply blood to the capillaries. This response involves in the first stage a shifting of the blood flow to the vital organs (i.e. reduced flow in the skin). As shock progresses into the second stage under-utilized capillaries in these organs are recruited for further blood flow. During the first two stages the body is successful in maintaining O₂ balance. It is for this reason that vital signs such as blood oxygenation and pH fail to detect the loss of blood volume. Significant delays in detection by vital organ tissue pO₂ measurements have been observed, and while this approach is promising, it still has not achieved satisfactory levels of correlation with major organ failure and morbidity. It is because of the critical role that capillaries play in first two stages of shock, in particular the early redistribution of flow away from peripheral tissue that our invention measures cutaneous capillary blood flow as an additional crucial parameter for the early indicator of the onset of hemorrhagic shock.

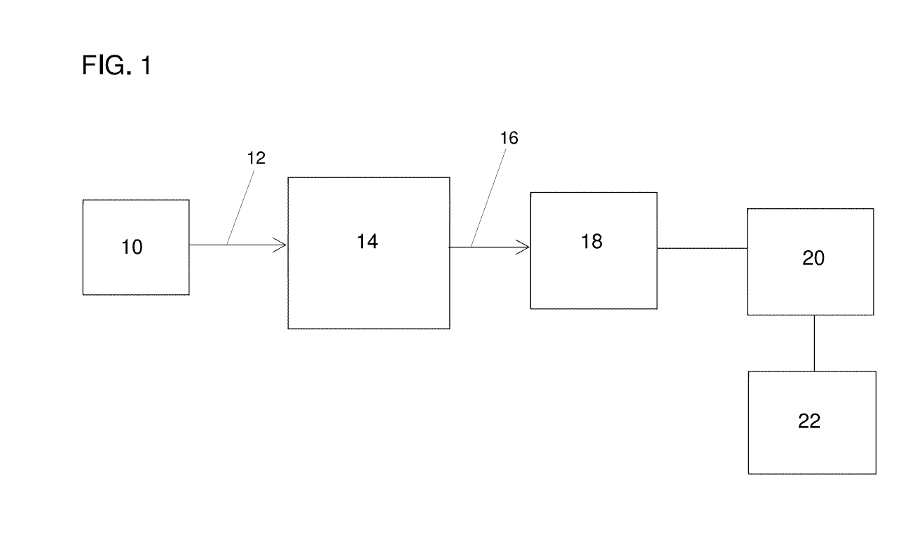

One embodiment of the hemodynamic monitoring system is schematically illustrated in FIG. 1. The system includes a light source 10 which directs incident light 12 toward a region 14 on a patient's body. Light is scattered, for example by tissue and blood within that region, to produce scattered light 16. The scattered light is detected by a detector 18. Electrical circuitry 20 associated with the detector generates a signal representative of the intensity of the detected scattered light. As described further below, the electrical circuitry may, for example, be integrated with the detector, or otherwise arranged. The electrical signal is transmitted to a processor 22 which analyzes temporal fluctuations in the signal. As described above, the analysis can be used to monitor hemorrhagic shock. In some embodiments, the analysis can be used to measure graft vascularization (or angiogenesis) of tissue grafts, for example, in buried flaps and/or once grafted. In other embodiments, the analysis may be used to measure tissue hypoxia at the interface between soft tissue and bone due to pressure. In some embodiments, the analysis is used to measure blood oxygen in addition to blood flow by simultaneously measuring fluctuations at multiple wavelengths of light. In some embodiments, the analysis may be used to assess tumor angiogenesis or monitor perfusion in burns. It should be understood that other uses are possible. In some embodiments, the methods described herein are useful in measuring flow and diffusive motion in in vitro settings, i.e. flow through capillary tubes, under conditions of single, multiple, and diffuse scattering. Flow in blood vessels beneath skin and in tissues is an example of diffusive scattering.

Light source 10 may be any suitable source of light, or multiple sources of light. For example, suitable light sources can include a laser (e.g., a temporally stabilized laser emitting visible and/or near infrared light), an LED, a lamp, or combinations thereof. The light source can be a semiconductor-based device. In some embodiments, the light source emits coherent light.

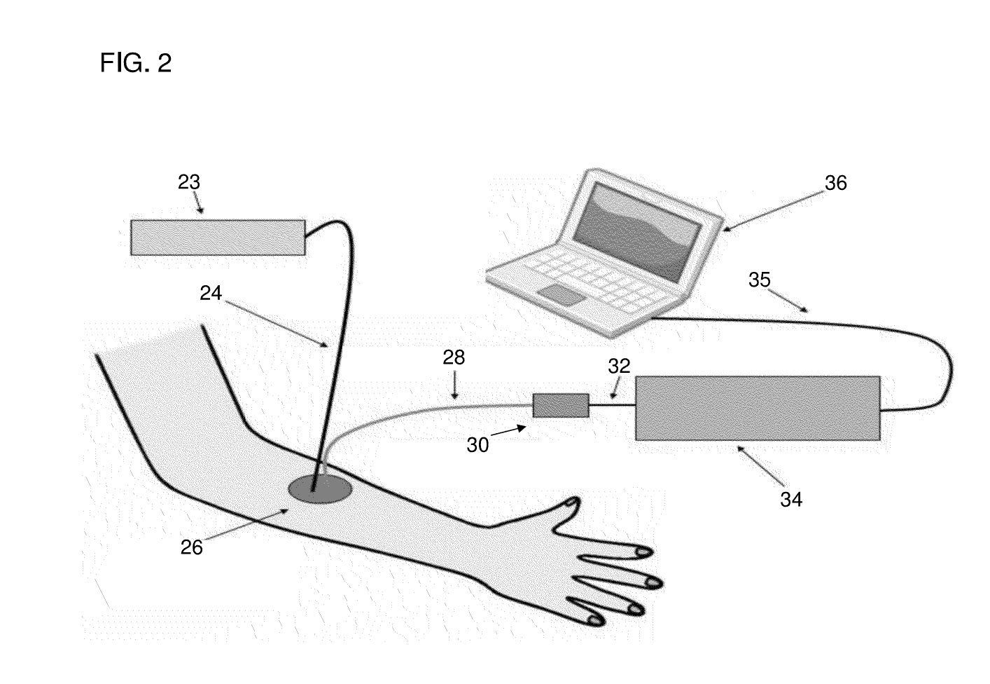

In some embodiments, though not all, incident light 12 may be directed to the region on the patient using a fiber optic (not shown in FIG. 1, shown in FIG. 2). The fiber optic may, for example, be a multimode fiber optic; or, in some cases, a single mode fiber optic. In some embodiments in which a fiber optic is not used to transmit incident light, the light source may be positioned near, or attached to, the body.

In some embodiments, though not all, scattered light 16 may be directed to the detector using a fiber optic (not shown in FIG. 1, shown in FIG. 2). In some embodiments, it is preferred for the fiber optic that transmits the scattered light to be a single mode fiber optic. Other embodiments may use multimode fiber optics. In some embodiments in which a fiber optic is not used to transmit scattered light, the detector may be positioned near, or attached to, the body.

In general, detector 18 may include any suitable component for detecting the scattered light and generating a resulting electrical signal. For example, suitable detectors include photodiodes, avalanche photodiodes (APDs), Geiger mode avalanche photodiodes (GPDs), photomultiplier tubes, solid state photomultipliers (SSPM), and CMOS device detectors. In some embodiments, more than one detector is used; and, in some cases, more than one type of detector is used. The detector(s) may be arranged, for example, at defined distance(s) from the one or more light sources. In some embodiments, the light detector is a photon counting device. For example, a single photon counting device may be preferred. Single photon counting devices can be more sensitive and, for example, detect light at lower intensities. In some embodiments, the light detector can be an analogue photon measuring device.

Electrical circuitry 20 associated with the detector can be any suitable type of circuitry known in the art. As noted above, the circuitry generates a signal representative of the intensity of the detected scattered light. In some embodiments, the circuitry may be integrated with the detector, for example, on the same chip.

As noted above, the electrical signal from the detector is transmitted to processor 22. In some embodiments, the signal may be transmitted wirelessly (e.g., electromagnetic transmission, infrared transmission). In some embodiments, the electrical signal is transmitted via a suitable data cable.

In general, any suitable processor or multiple processors may be used. The processor(s) can be, for example, a microprocessor, a field programmable gate array (FPGA), an arithmetic logic unit, or any other suitable processing device. The processor may be in a single computer or distributed among multiple computers. Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device.

In some embodiments, the processor performs autocorrelation analysis. In some embodiments, the processor performs Fourier analysis or wavelet analysis or analysis of pulse height distributions. In some embodiments the processor combines analysis of temporal fluctuations with other vital monitors available to the physician and combines these using “smart multiparametric analysis” such as principal component analysis. These additional parameters may include but are not limited to gastric or sublingual pH measurement, near-infrared reflectance oximetry, heart rate or pulse, respiratory rate, beat-to beat heart rate variability, and acoustic arterial flow analysis.

In some embodiments, though not shown, the system includes cooling mechanisms which may cool the detectors and/or the light sources during use. For example, the cooling mechanism may thermoelectrically cool these components. Cooling may increase stability and reduce noise.

FIG. 2 illustrates another embodiment of a hemodynamic monitoring system. This embodiment includes a light source (e.g., stabilized laser) 23 which directs light into a first fiber optic (e.g., multimode fiber optic) 24 which transmits the incident light to a sensor patch (or housing) 26 affixed to the skin of a patient. A second fiber optic (e.g., single mode fiber optic) 28 is also connected to the sensor patch and collects the scattered light from some distance away from the first fiber optic. The second fiber optic transmits the light to a light detector 30 which, in this embodiment, includes integrated electrical circuitry which can enable operation of the light detector and initial processing. A data cable 32 from this circuitry transmits the signal from the measurement circuit to a processor 34 for analyzing intensity fluctuations. In some embodiments, the processor performs autocorrelation analysis. In other embodiments, it performs Fourier analysis or wavelet analysis or analysis of pulse height distributions. Data from the processor may be taken via a cable or wirelessly, preferentially a USB cable 35 or fire-wire cable, to a laptop computer 36 or similar microprocessing device for further analysis and processing.

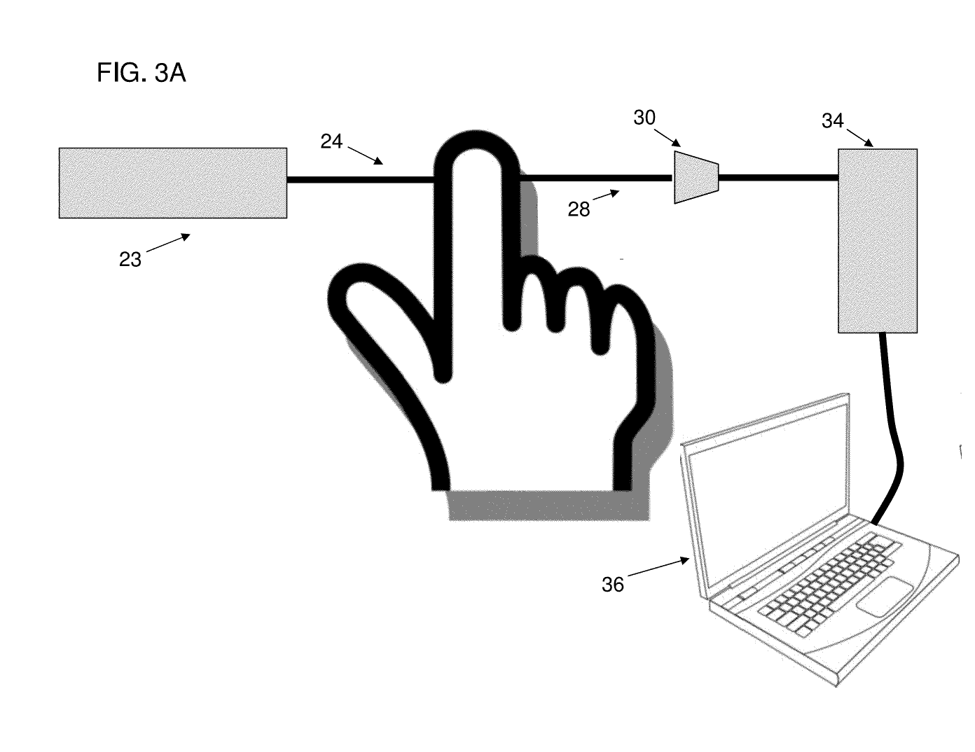

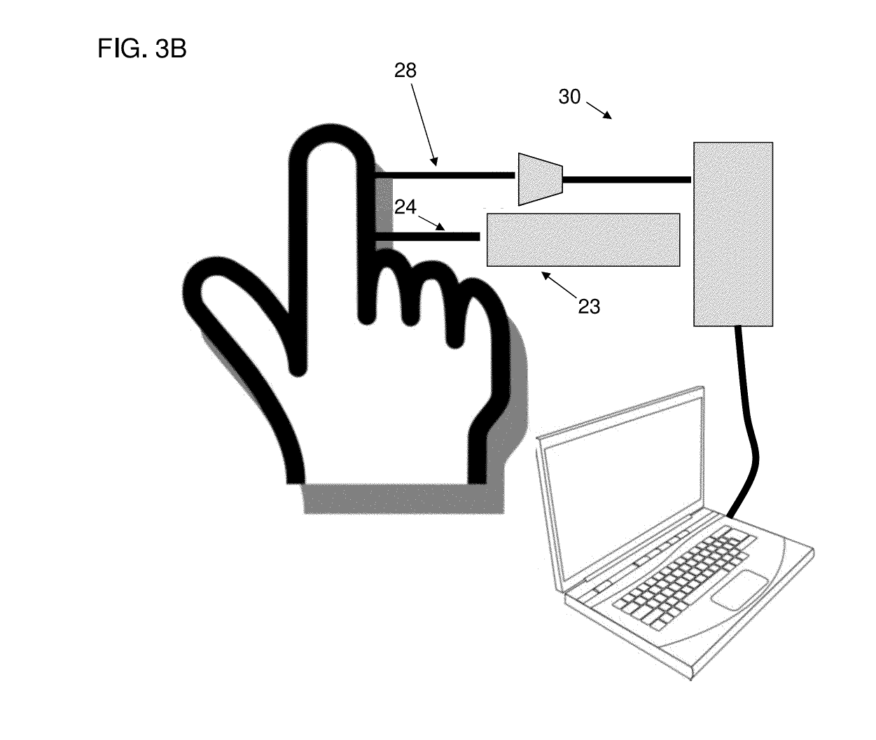

FIG. 3A illustrates an embodiment in which the hemodynamic monitoring system is operated in a transmission mode. Transmission mode is defined as the case where the angle between the mean input light path and the output light path is greater than ninety degrees, thus resulting in measurement of forward light scattering.

FIG. 3B illustrates an embodiment in which the hemodynamic monitoring system is operated in a reflectance mode. Reflectance mode is defined as the case where the angle between the mean input light path and the output light path is less than ninety degrees, thus resulting in measurement of back light scattering.

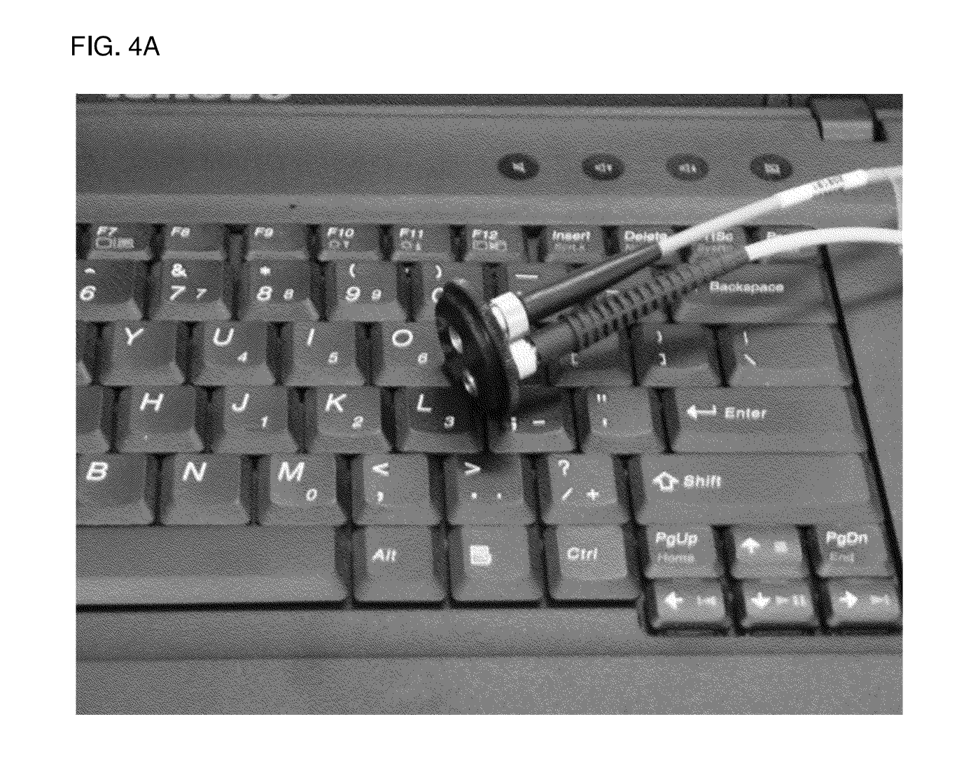

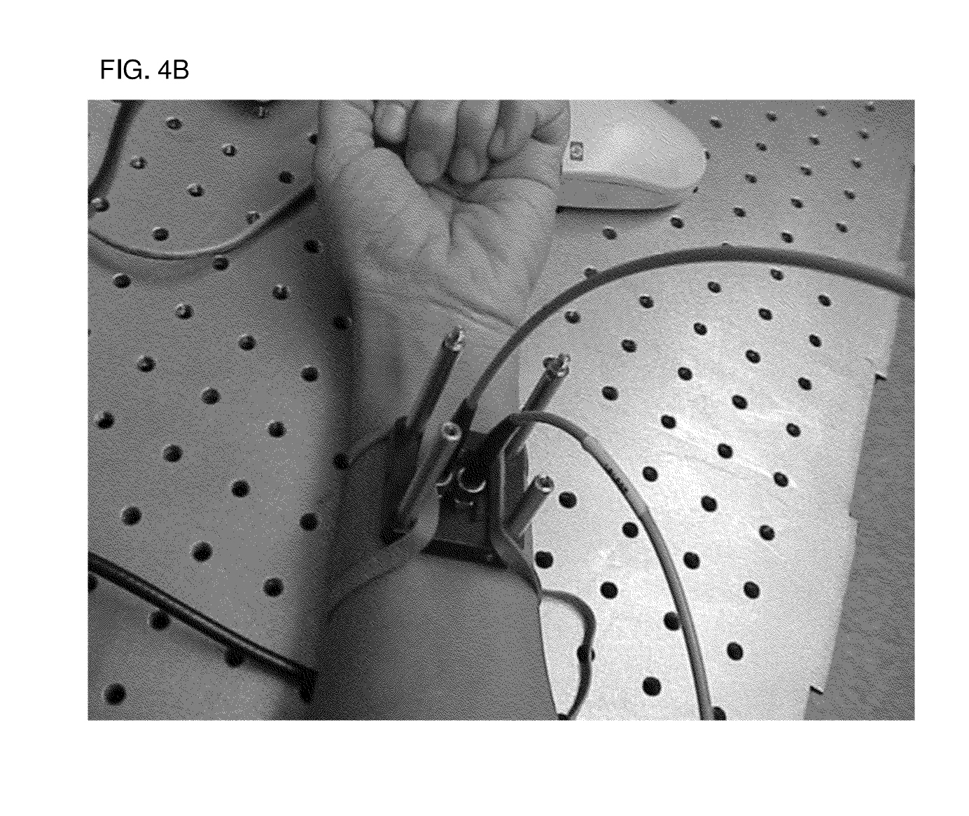

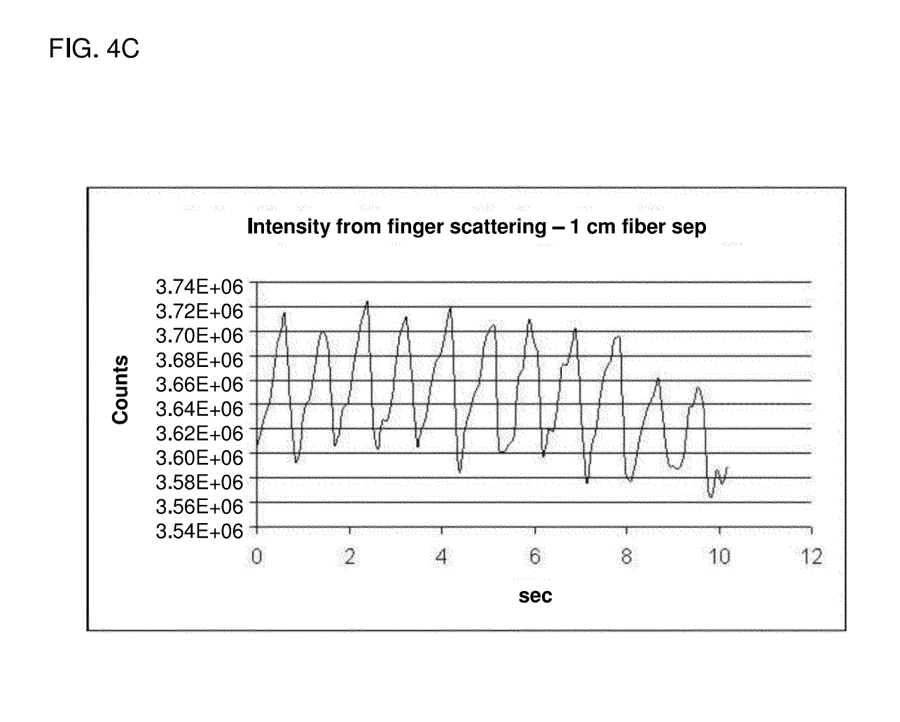

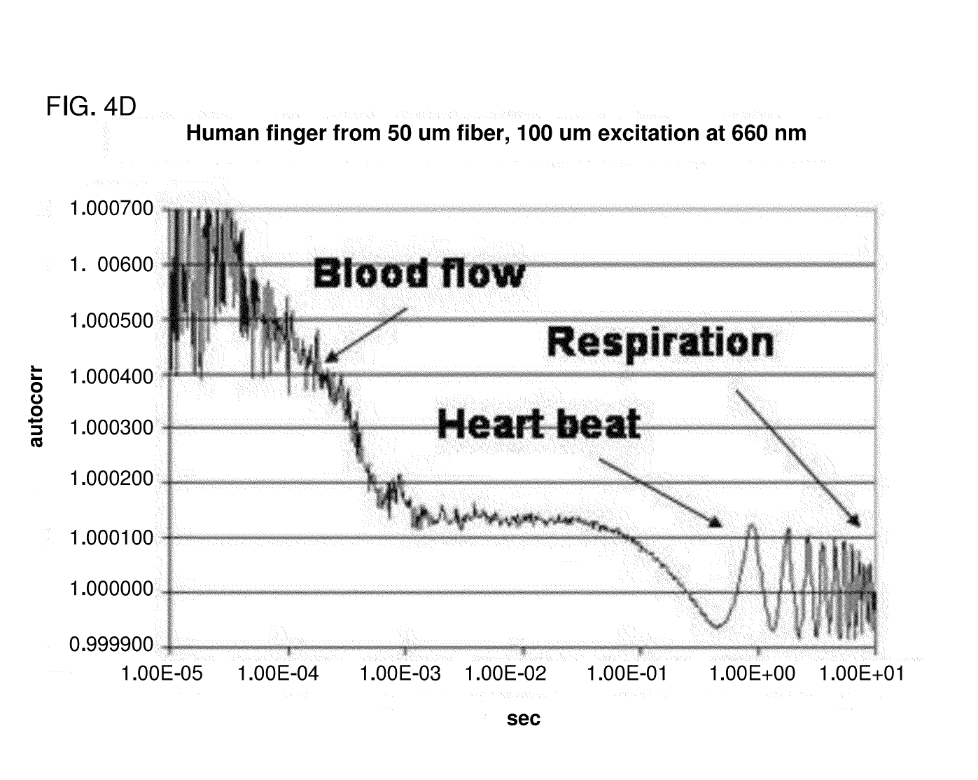

FIG. 4A shows a close-up of a sensor patch according to an embodiment with the input and output fibers. FIG. 4B shows such a sensor patch attached to the skin of a patient with a strap. In certain preferred embodiments, attachment is accomplished with an adhesive.

FIG. 4C shows intensity data taken with such a patch where the separation distance between input and output fibers is 1 cm. Three sources of intensity fluctuation are observed: rapid, us time scale, fluctuations due to mean blood flow, oscillatory fluctuations, of −1 sec, duration due to heartbeat, and a slow undulation, of −10 to 20 sec duration, due to respiration. These are clearly distinguished when the autocorrelation of the data of FIG. 4C is calculated and plotted in FIG. 4D.

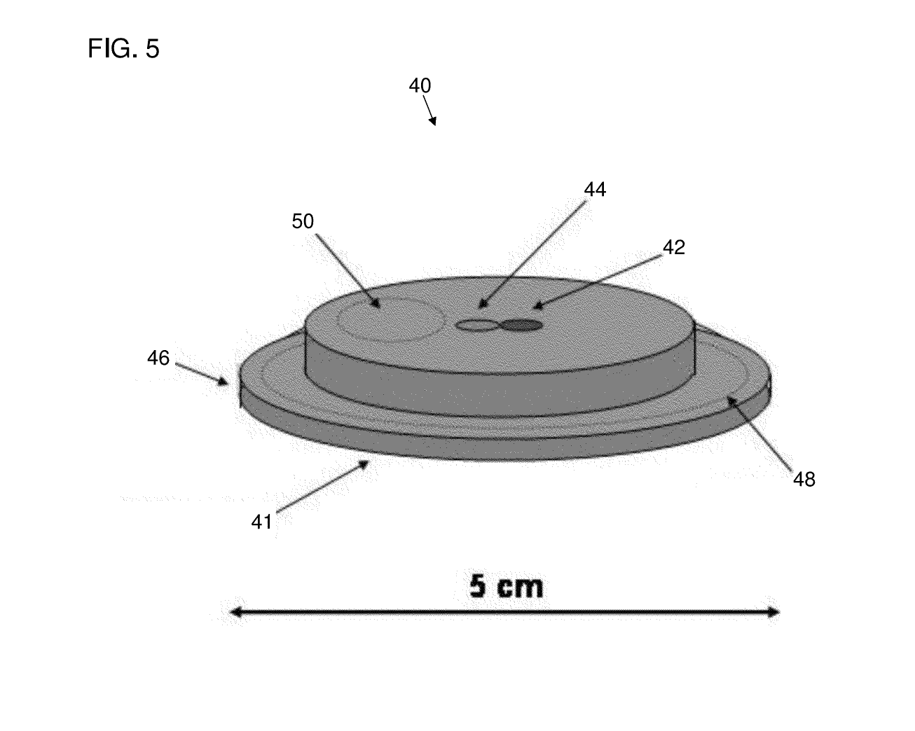

One embodiment of an integrated hemodynamic monitoring device is shown in FIG. 5. In this embodiment, the device includes a sensor patch in the form of a housing 40 which may be mounted to the skin. For example, the device may be held against the skin using a strap, or attached to the skin using an adhesive layer 41. In this embodiment, a light source 42 (e.g., laser diode) and a detector 44 (e.g., CMOS detector with integrated electronics) are integrated with the housing, and each other. It should be understood that there can be more than one light source and or detector, and the separation distances can be different to enable simultaneous measurement of multiple depths.

In this embodiment, the housing may be relatively compact, for example, having a volume of less than 10 cm³.

The housing may be formed of any suitable material including polymeric materials. In some embodiments, the housing may be formed of a flexible material so that the housing may conform better to the body. The housing may have a base portion, as shown. In some embodiments, the base (and, in some cases, other portions of the housing) is formed of a clear plastic to enable light transmission. The housing, or portions thereof (e.g., base), may be designed to be disposable. For example, the housing, or portions thereof (e.g., base), may be formed of a disposable plastic.

In the embodiment of FIG. 5, the adhesive layer may be any type of suitable adhesive. For example, the adhesive may be glue, double-sided sticky tape, amongst others. The light source may be a laser diode, or other suitable light source described above. The detector may be a CMOS detector, or other suitable detector described above. The detector may have integrated electronic circuitry which support device function and process the electrical signal. For example, the electronics may be integrated as part of a CMOS chip. The device, as shown, includes a wireless transmitter and antenna 48 that communicates with a remote processor that need not be integrated with the device (e.g., processor 22 described above). This embodiment includes a battery 50 also integrated with the housing and other components to provide power to other components on the device such as the light source, electronic circuitry and transmitter. In some embodiments, the device may include a thermoelectric cooling mechanism (not shown) integrated with the housing which may increase stability and reduce noise. Additional stabilization can also be provided by electronic circuitry in the device, to correct for other sources of noise such as light source fluctuations, ambient signals, and electrical noise.

The following examples are illustrative of embodiments of the invention but should not be considered limiting in any way.

### EXAMPLES

### Example 1

### Measuring Fluid Flow in a Tube Using Transmission Mode

In a first example a laboratory prototype that was used to measure the required physiological parameters simulated in a phantom. In this setup we used two diode lasers (wavelength 660 nm and 980 nm) to illuminate the target. The phantom comprised: a diffused plastic tubing that had an inner diameter of 0.8 mm and a wall thickness of −1 mm, and a diffused phantom made of resin with a cylindrical bore as a blood conduit.

The blood flow rate through the tubing was adjusted using the pump settings. Initial calibrations were performed using a syringe pump to generate constant velocity flow. Subsequent measurements were made using a peristaltic pump to simulate natural blood. Oxygenation was measured using a calibrated dissolved-oxygen sensitive platinum electrode.

The lasers were focused to a spot of approximately 100 μm inside the tube. The sources could, in principle, be placed against the target, as with conventional pulse oximeters. The scattered light (both transmitted and reflected) was collected and the technique tested using single mode and multimode fibers. A 980 nm single mode fiber (6 μm in diameter) or a 50 μm multimode fiber was placed close to the phantom.

Reflection measurements were tested against transmission measurements and the two displayed similar behavior. The signal from the fiber was detected using a Perkin Elmer (PE) (Salem, Mass.) single photon avalanche photodiode (SPCM). The SPCM is thermoelectrically cooled and temperature controlled for stabilized performance. The SPCM outputs a digital pulse for every detected photon, which is fed to a correlator.com hardware correlator with 12.5 ns resolution, that is interfaced to a computer (Flex-08). The SPCM has a dead-time of 100 ns, which determines the achievable resolution in our measurements. We compared the APD devices with photodiodes (PD) during oxygen measurements. For direct measurements, the detector was placed against the tube separated by an aperture.

Most dynamic scattering experiments rely on the intensity beating of scattered signals either from two different particles (homodyne method) or beating of reference and a scattered wave (heterodyne method), which provides information about the process dynamics. For this program we adopted a similar approach to measuring blood parameters—called the intensity correlation approach. In our approach, the intensity autocorrelation of the time-series signal is measured as a function of time delay to determine the flow velocity as well as other dynamics of the system such as the pulse rate.

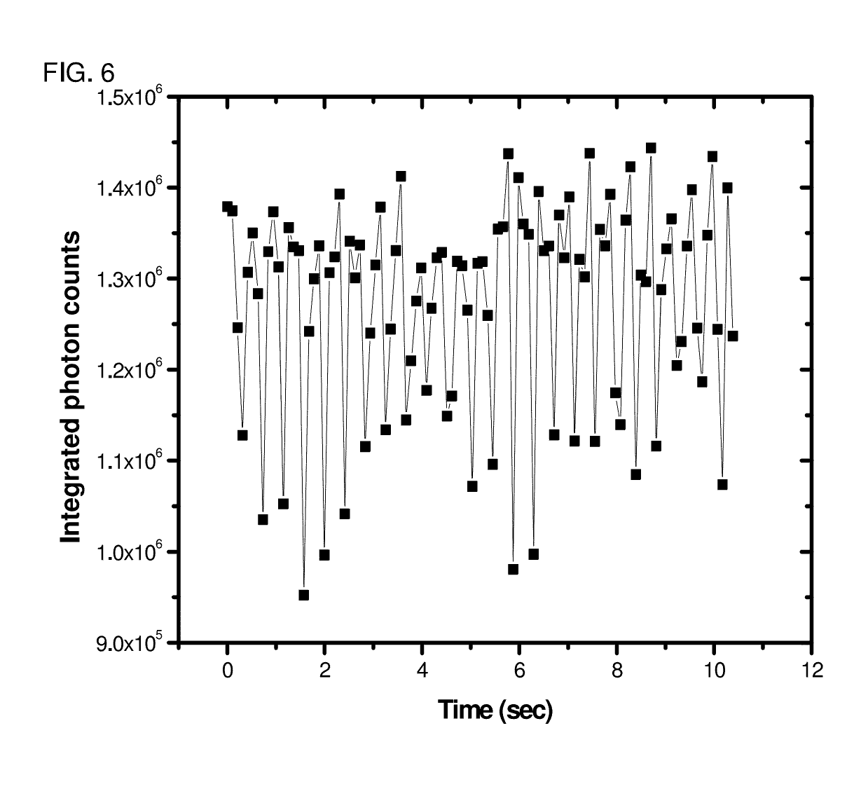

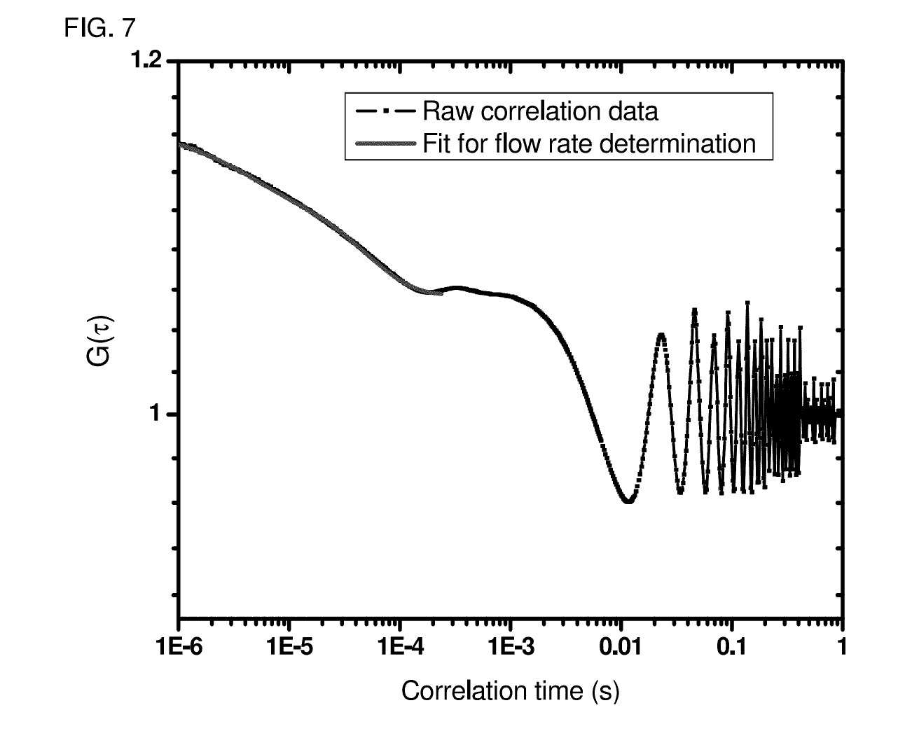

FIG. 6 shows an example of the raw intensity data as a function of time taken with 980 nm laser illumination on blood flowing through a tube pumped using a peristaltic pump (transmission mode). The intensity is the time integrated photon count from the single photon counter over a period of 100 μs. As can be seen from FIG. 6, the intensity is an oscillatory function in time due to the peristaltic nature of the flow. FIG. 7 shows the corresponding autocorrelation data that is obtained from the intensity data. From FIG. 7 it can be seen that the correlation signal has two components: one is the exponential decay that occurs at earlier correlation times (˜10 μs) and the other is an oscillatory component that arises due to pulsatile nature of flow through the tube (˜0.03 seconds). The intensity data is intrinsically noisy. However, because of the way that correlation analysis averages data, it is efficient at extracting these two key parameters from the intensity profiles. The first exponential component can be fitted to obtain the flow rate or the blood velocity (This is the average blood velocity). The frequency of the second component can be used to obtain the pump rate or heart or pulse rate.

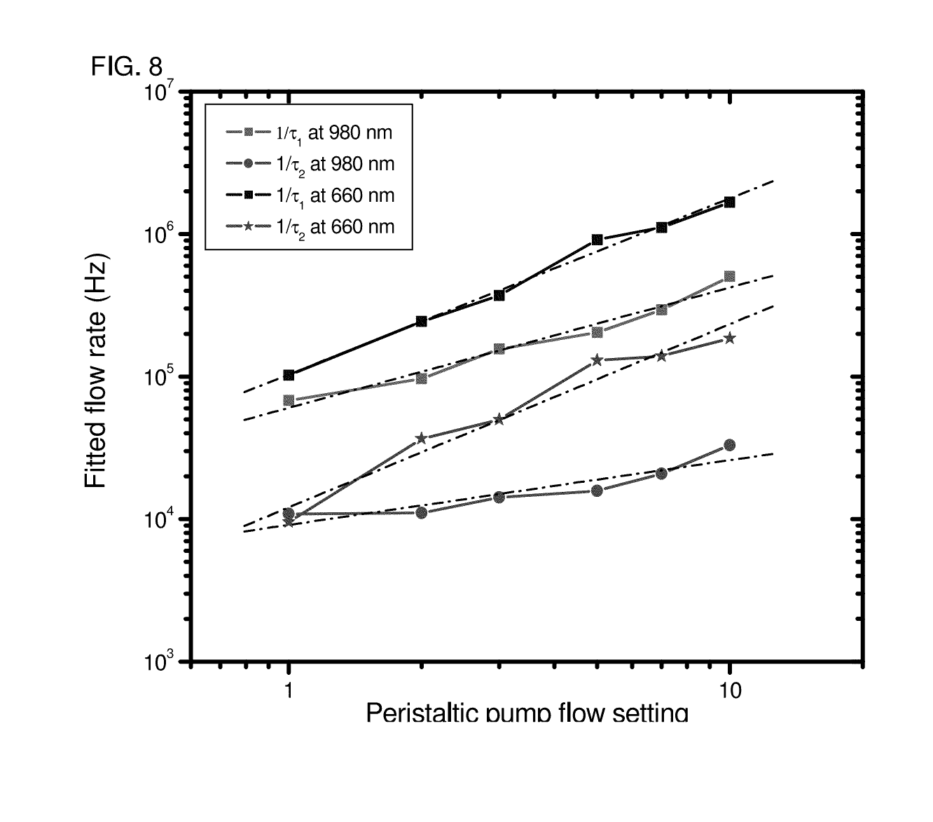

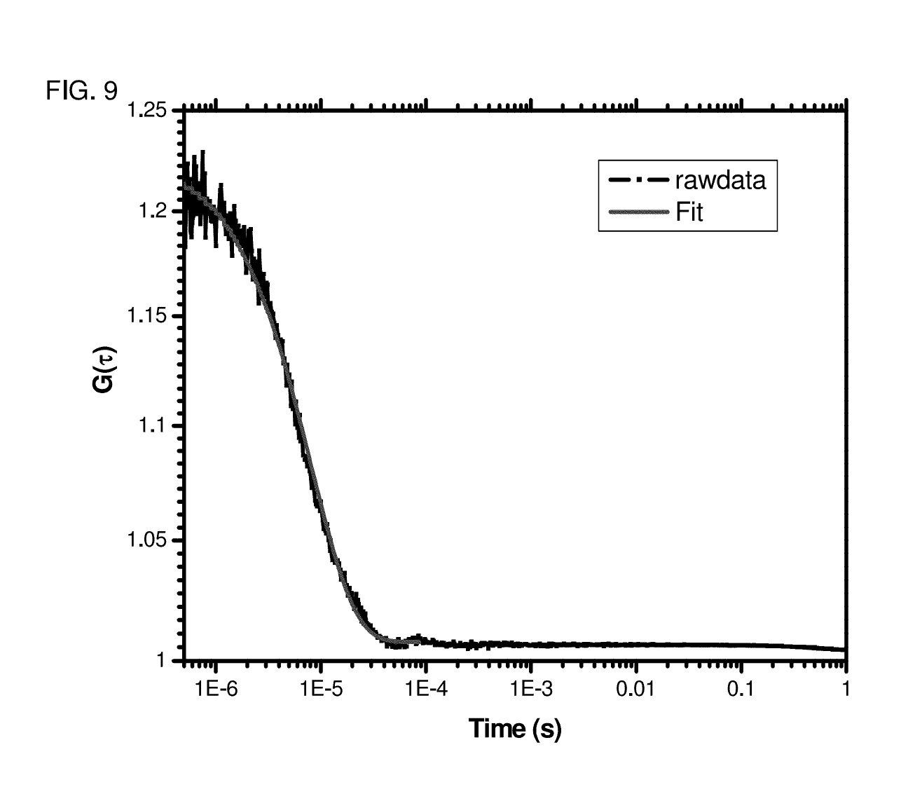

FIG. 8 and FIG. 9 show the raw data and the autocorrelation plots, respectively that are obtained using a reflection mode setup for the case where the flow rate through the tube is set to a high value using the peristaltic pump. In the case of low velocities, the flow through the tube is axial and laminar in nature. For such homogeneous shear flow velocities and for low flow speeds, the correlation function decays, as predicted from theory, with a Gaussian time dependence rather than simple exponential time dependence found for higher flow speeds. This is due to the fact that in a shear flow the separation between pairs of particles grows linearly in time unlike the square root of time dependence in the case of diffusion. Hence, for the slow flow rate case, the data can be fit to a second order exponential decay of the form exp−(t/τ)², where τ is the fitting parameter that can be used to obtain the flow velocity.

In the case of higher flow rates, the nature of the flow is more non-axial in the sense that there are more velocity components compared to the slow flow axial component (more diffuse nature of flow). This high flow rate case the fitting function will be a first order exponential or a combination of two first order exponentials of the form, A exp−(t/τ₁), B exp−(t/τ₂). Two values for τ imply that there are two velocities involved in the peristaltic flow. Such a dual velocity behavior in peristaltic flow has been reported before, where the blood flow velocity was measured using a laser Doppler vibrometer (LDV) at the carotid artery. For flow settings in the moderate flow rates a combination of the two cases is used to fit the initial decay in the correlation plot and is of the form, A exp−(t/τ₁)², B exp−(t/τ₂)². An example of a fitting to the correlation plot is shown in FIG. 3 by the red curve, for the period between 1 μs and 0.5 ms for a high flow rate using a peristaltic pump. In FIG. 8 we plot the inverse of the fitted time constants that we obtained for the peristaltic flow correlation data for different flow settings. For each flow setting we obtained two time constants, a fast component τ₁ and a slower component τ₂. For a given wavelength, both τ₁ and τ₂ change with nearly similar slopes as the flow settings are varied. This can be seen from FIG. 8, where the slopes for τ₁ and τ₂ are almost identical. The y-axis can be correlated to the actual flow velocity after calibrating the flow settings on the peristaltic pump using measurements performed with syringe pump. The fitting parameters, τ obtained from the syringe pump data can then be used to create a look-up table for different flow velocities.

### Example 2

### Calibrating Flow Rate in a Tube

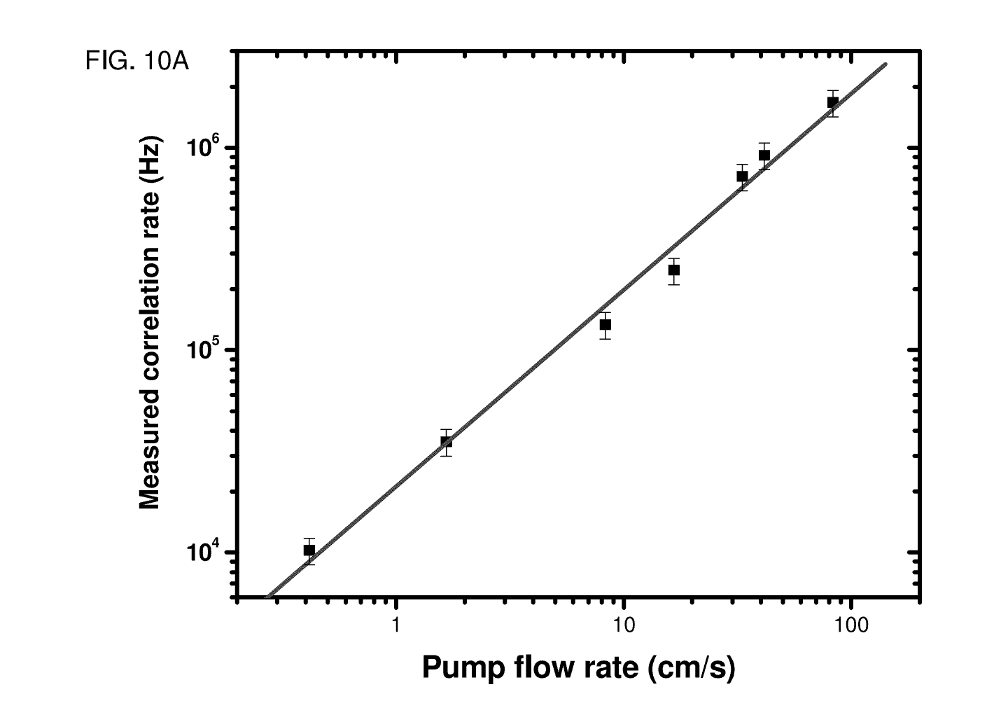

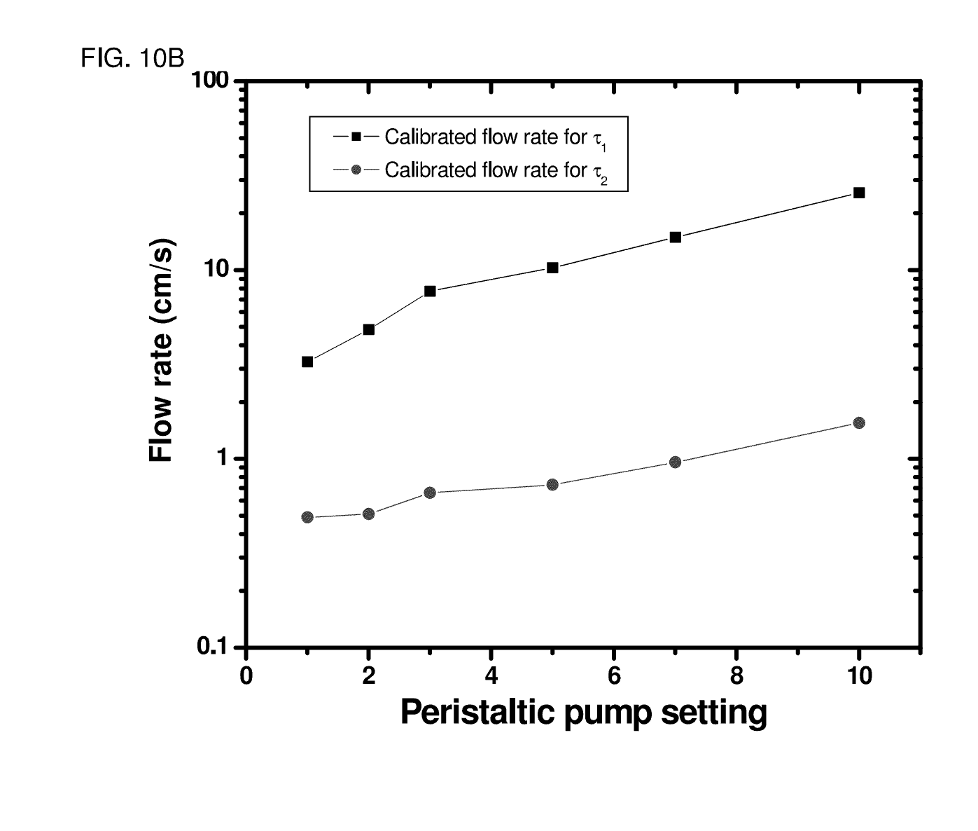

In order to calibrate the fitted flow rate for the actual blood flow velocity, we performed the same set of measurements using blood pumped by a syringe pump instead of the peristaltic pump. In this case, the flow rate is uniform as a function of time and can be measured accurately by measuring the volume of liquid flowing out of the tube in a fixed time. We obtained correlation plots for different flow rates using a syringe pump, and FIG. 9 shows an example of one such correlation plot. As can be seen, the profile for short correlation times looks similar to that of the constant flow. However, the oscillations at longer times are absent, as expected in this case. Also shown, is the fitted flow rate by the red line superimposed on the raw data black dotted line. The fitting is performed using the same procedure as mentioned above for the peristaltic pump data. The velocity calibration is obtained by plotting the reciprocals of the fitted ti's as a function of the flow rate and is shown in FIG. 10A. The measured data can be fitted to a straight line as shown by the red line with a slope of (6.85±0.26)×10⁴ cm⁻¹.

Using this calibration plot, we can obtain the flow rate for the peristaltic flow measurements by fitting to the correlation plots for different pump settings. In FIG. 10B, we plot the flow rate obtained from the calibration plot for different pump settings. As can be seen we obtain two different flow rates for each pump setting, shown by the black and red symbols in the plot corresponding to the two velocity components discussed earlier.

### Example 3

### Measuring Oscillatory Flow

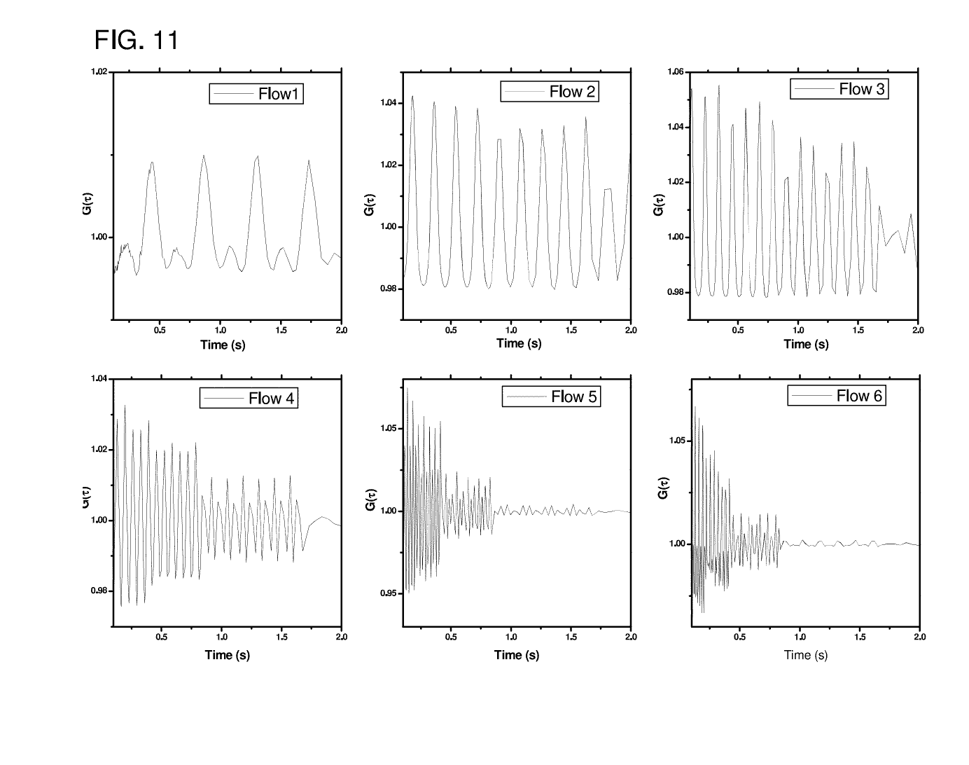

We can also obtain the heart rate or the pulse rate from the correlation plot in the longer times range. A unique feature of the peristaltic pump is that the flow velocity is determined by the pulse rate. In FIG. 11, we show correlation data in this time range for different flow rates that are increasing respectively from Flow 1 to Flow 6. As can be seen clearly, the oscillation frequency increases with flow rate and also shown in FIG. 9 is the plot of the measured oscillation frequency or measured pulse rate for different flow settings.

### Example 4

### Measuring Oxygen Saturation

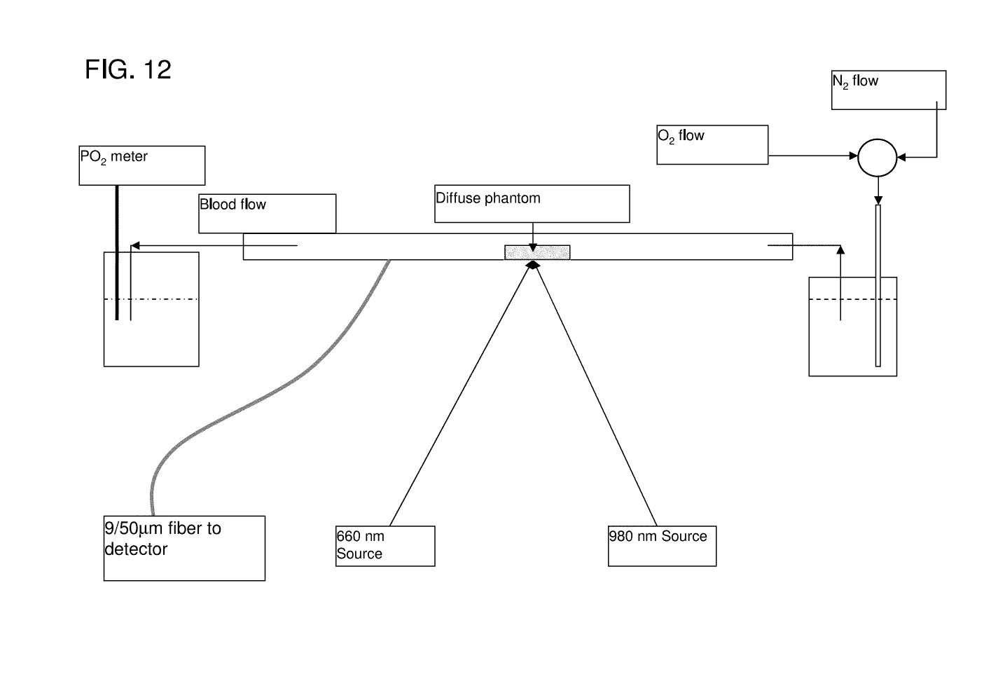

The ratio of the oscillation amplitude both in the correlation function and in the raw data (at two wavelengths corresponding to minimum and maximum hemoglobin absorption) can be used to obtain the blood oxygen saturation. This parameter is significant in the identification of hypoxia or loss blood oxygen saturation and occurs for example in hypoxic hypoxia, hemorrhagic shock, stroke, pressure ulcerations, and at the site of neoplastic tumors. In our approach, this can be obtained from the same correlation analysis performed on the intensity data obtained with two different wavelengths. FIG. 12 shows a schematic of the experimental setup used for the oxygenation measurements (reflection mode). This measurement is performed using two or more wavelengths (corresponding to the minimum absorption at 660 nm for oxy-hemoglobin and at 980 nm for deoxyhemoglobin) in order to obtain a baseline measurement for quantitative estimates of the oxygen saturation. It can be shown that, in the autocorrelation signal, the amplitude of the oscillations carries information about the hemoglobin absorption. The oscillatory component in the autocorrelation signal, corresponding to the pulsatile nature of flow, are of the form A²/2B, where A is the required amplitude that changes with hemoglobin absorption and B is the average intensity of the measured signal. Using the correlation function improves our ability, compared to measuring intensity alone as for instance in pulse oximetry, to discriminate the regular pulsatile amplitude from other sources of noise in the signal including: body motion, respiration, and aircraft motion. We performed experiments to measure the oxygenation at two different wavelengths as a function of oxygen concentration. To accomplish this, we varied the dissolved oxygen concentration by bubbling a mixture of oxygen and nitrogen through the blood reservoir, while monitoring the value using a dissolved oxygen meter (ISO₂, World Precision Instruments). This value gives the total dissolved oxygen concentration, PO₂.

The algorithm to obtain the blood oxygenation improves upon what is done in pulse oxymeters, and is further computationally complicated to evaluate in reflection mode measurement. It is assumed that the scattering properties of the blood and tissue do not change significantly as a function of wavelength of excitation. We performed experiments to measure the oxygenation at two different wavelengths as a function of oxygen concentration. Oxygenated and deoxygenated hemoglobin have different absorbance spectra and this property is used to measure the relative concentration of oxygenated hemoglobin. One could either measure the total signal at each wavelength or one could track the DC and the AC component separately as is done in pulse oximeters. Here, we have done the former, i.e., simply track the total signal at each wavelength.

The amplitude of the oscillatory components are proportional to how much scattering and absorption the light has experienced while traveling through the phantom tissue. Signal for oxygenation is stronger than that for deoxygenation as expected, since light at 660-nm is minimally absorbed by oxygenated Hb. This is also confirmed by the increased average intensity for oxygenated blood in the Ratios of Signal₆₆₀/Signal₉₉₀ provides us with the calibration plot necessary to create a lookup table as a function of PO₂.

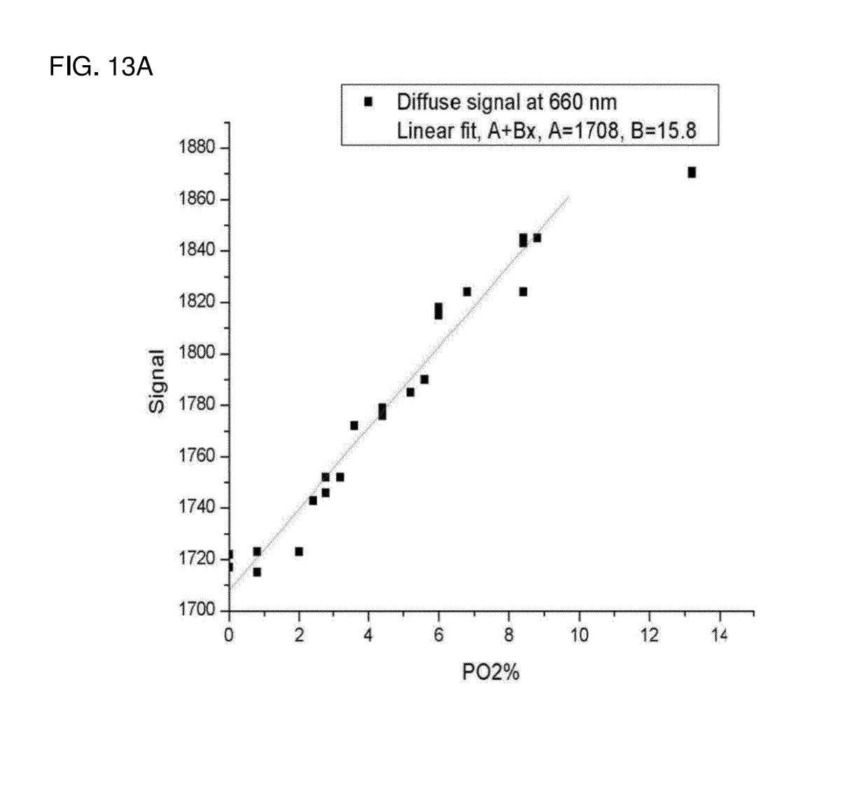

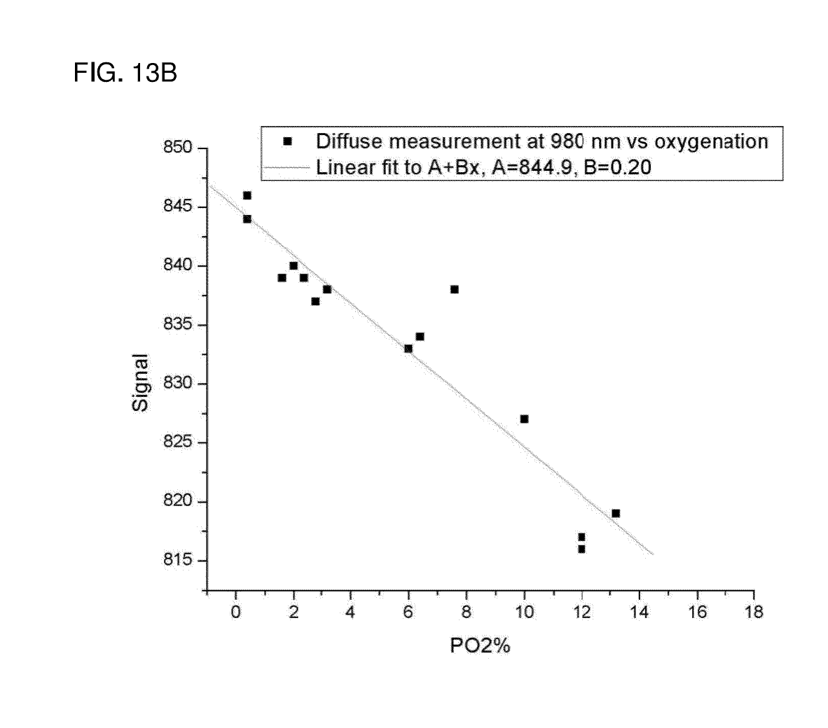

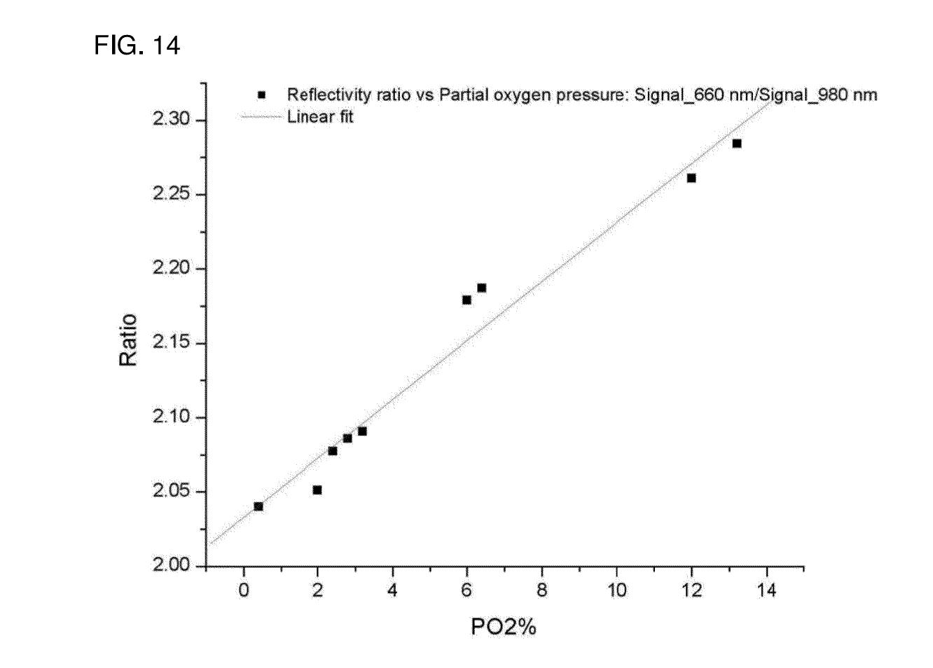

FIGS. 13A and 13B show the response of oxygenation of blood at two wavelengths. To demonstrate that the photon counting detector measures signal similar to that obtained with a pulse oximeter, which employs analog detection with photodiodes, we also installed a photodiode to measure the signal at simultaneously. The inset in FIG. 13A, shows a good correlation between the APD and the photodiode outputs. FIG. 13A and FIG. 13B show the signal response at 660 nm and at 980 nm respectively. Ratios of Signal₆₆₀/Signal₉₉₀ (FIG. 14) provide us the calibration plot necessary to create a lookup table as a function of PO₂. PO₂ values can be further converted to blood oxygenation levels using the established blood saturation data.

### Example 5

### Measurements of Blood Flow in Humans

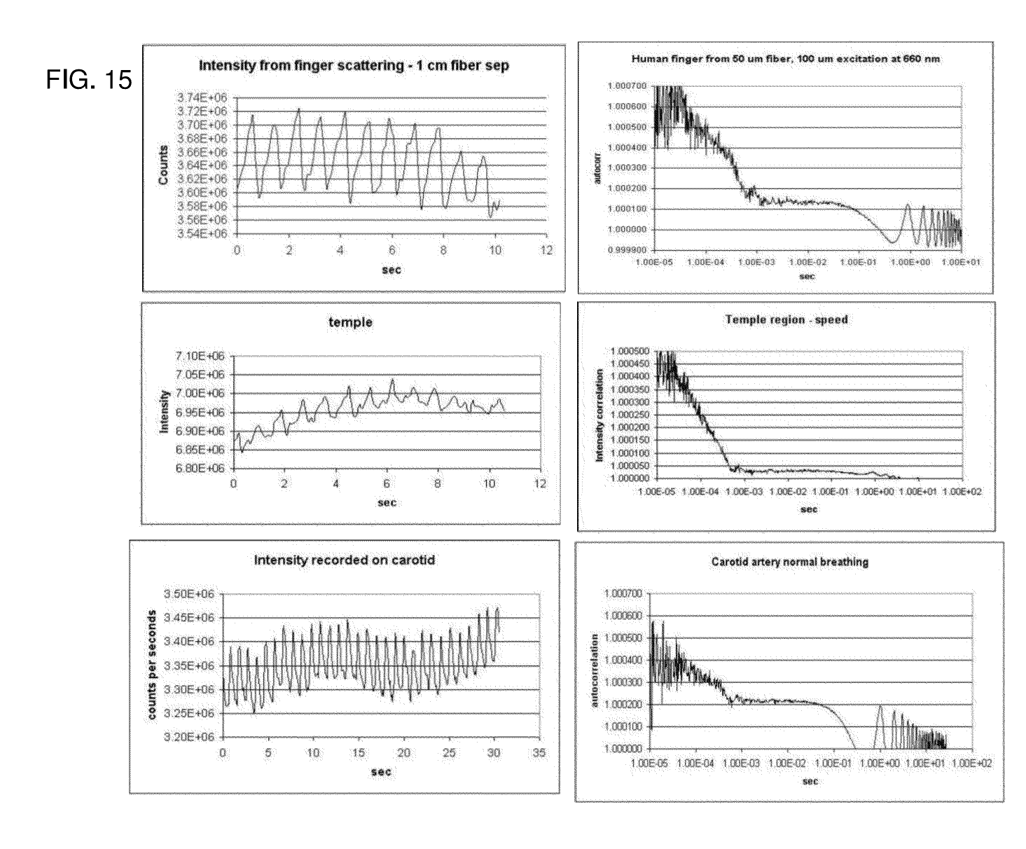

We investigated a geometry (see FIG. 4) that mimics the final prototype design, where a patch on the pilot's forehead near the superficial temporal artery contains the lasers and the detectors. In FIG. 15, we show data obtained in the reflection geometry from a person's finger, temple and neck region. The plots on the left shows the intensity data and the plots on the right show the correlation data obtained from the intensity data. The intensity data provides the heart rate, while correlation data provides much more information. The signal decay in the first few 100 μs indicates blood velocity. The oscillatory components in the range of seconds provide us the heart rate. Slow changes at tens of seconds provide us the respiratory rate.

### Example 6

### Measurement of Respiratory Rate in Humans

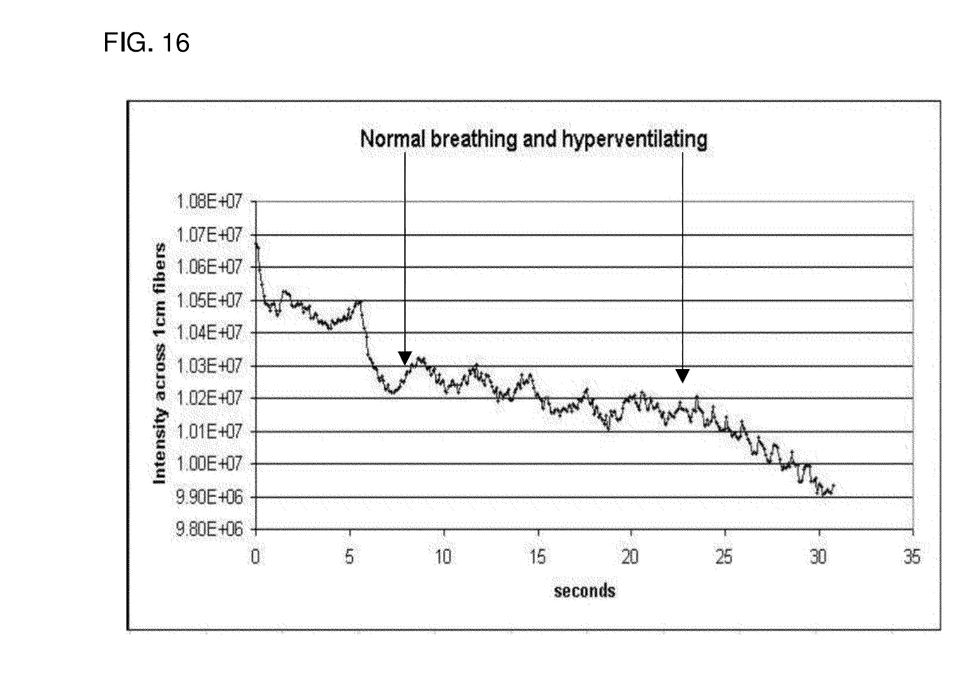

To demonstrate that our device can detect and measure respiratory rate, the subject first performed normal breathing and then subsequently hyperventilated. Our device while measuring the signal from carotid artery (see FIG. 16) cleanly picked up both normal and hyperventilated signals.

### Example 7

### Distinguishing Between Arterial and Capillary Blood Flow

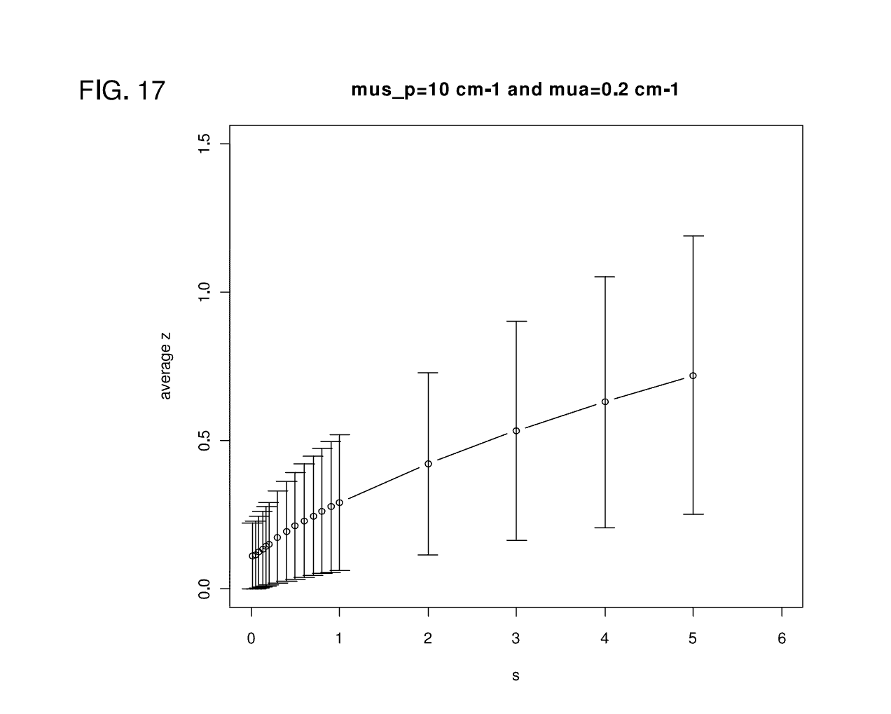

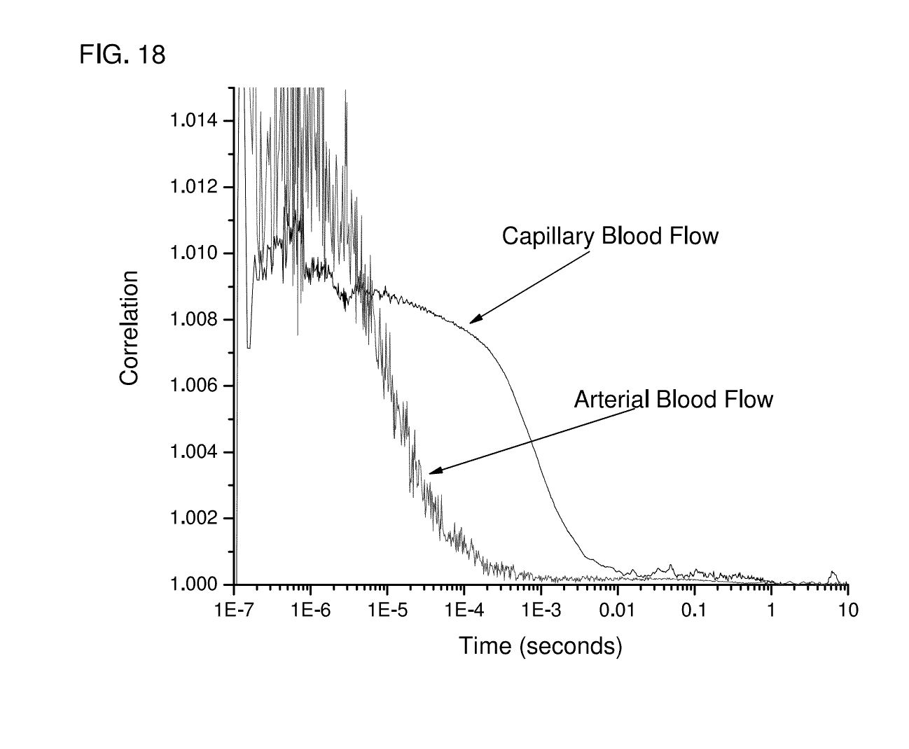

The depth within the tissue that one is monitoring may be controlled by changing the separation distance between the source and the detector. This may be modeled using either light diffusion theory or a Monte Carlo approach. FIG. 17 shoes such a calculation based upon light diffusion in tissue for the case where the scattering coefficient μₛ is assumed to be 10 cm⁻¹ and the absorbance coefficient μₐ is assumed to be 0.2 cm⁻¹. The average depth monitored, z, is shown as a function of source-detector separation distance, s. The bars indicate the 68% confidence interval. The functionality of this selection approach is shown in FIG. 18 that compares the short time autocorrelation function for blood flow in a fingertip where we have separated the input and output fibers by 1 cm, labeled arterial blood flow, and 2 mm, labeled capillary blood flow. As the fibers are moved closer and closer to each other the device preferentially monitors signal from progressively shallower distances. Thus, by using a 2 mm separation, the inventors can focus on measuring the cutaneous, capillary blood flow.

What is claimed is:

1. An integrated device for assessing blood flow in tissue of a patient, wherein the device is configured to be mounted to the patient, the device comprising:
a housing;
a light source integrated with the housing, the light source constructed and arranged to direct light toward a region in the patient including tissue in which blood flows; and
a single photon counting light detector integrated with the housing, the light detector constructed and arranged to detect photons of light scattered by the tissue and the blood and to output a single digital pulse for every detected photon.

2. The device of claim 1, wherein the housing has an outer surface, and the light source and the light detector are positioned on the outer surface.

3. The device of claim 1, further comprising a battery electrically connected to the light source to provide power to the light source.

4. The device of claim 1, wherein the light source is semiconductor-based.

5. The device of claim 4, wherein the light source is an LED or laser diode.

6. The device of claim 1, further comprising signal processing electronics integrated with the light detector.

7. The device of claim 1, wherein the light detector is a CMOS-based device.

8. The device of claim 7, wherein electronic processing circuitry is incorporated into the CMOS-based device.

9. The device of claim 1, wherein the light detector is chosen from the group consisting of: photomultiplier tubes, charge coupled devices, solid state photomultipliers, silicon photodiodes, avalanche photodiodes and Geiger mode avalanche photodiodes.

10. The device of claim 1, wherein the housing has a volume of less than 10 cm³.

11. The device of claim 1, wherein the device further comprises an adhesive on a portion of the outer surface of the device.

12. The device of claim 1, wherein the device further comprises a wireless antenna associated with the detector designed to transmit signals representative of the scattered light intensity.

13. The device of claim 1, wherein the housing comprises a polymeric material.

14. A system for assessing blood flow in tissue of a patient comprising:
an integrated device for assessing blood flow in tissue of a patient, wherein the device is configured to be mounted to the patient, the device comprising:
a housing;
a light source integrated with the housing, the light source constructed and arranged to direct light toward a region in the patient including tissue in which blood flows; and
a single photon counting light detector integrated with the housing, the light detector constructed and arranged to detect photons of light scattered by the tissue and the blood and to output a single digital pulse for every detected photon thereby generating an electrical signal; and
a processor configured to analyze temporal fluctuations in the electrical signal to monitor for hemorrhagic shock in the patient.
